(19) United States
(12) Patent Application Publication
(10) Pub. No.: US 2006/0142633 A1
(43) Pub. Date: Jun. 29, 2006

(54) Blood pumping system and procedure

(76) Inventors: Rodney James Lane, Castlecrag (AU); John Campbell Woodard, Turramurra (AU); Anthony Philip Alder, Hornsby (AU)

Correspondence Address: Abelman Frayne & Schwab, 666 Third Avenue, 10th Floor, New York, NY, 10017-5621, US

(21) Appl. No.: 10/539,999

(22) PCT Filed: Dec. 17, 2003
(86) PCT No.: PCT/AU03/01679

§ 371 Date: Dec. 14, 2005

(30) Foreign Application Priority Data
Dec. 17, 2002 (US) 60434473

(51) Int. Cl. A61M 1/10

(52) U.S. Cl. 600/16

(57) ABSTRACT

A blood pumping system for supplementing distal blood perfusion comprising a blood pressure altering device in the form of a blood pump (1) having an inlet (10) and an outlet (9) which also includes an upstream end (2) and a downstream end (4). The blood pressure altering device (1) cooperates with a circulatory system of a patient (15) to promote blood flow throughout at least one or more distal regions including limbs, a brain region of a patient by altering vascular blood pressure. The blood pressure altering device (1), when in use, is positioned in series with the normal blood flow of the circulatory system of a patient (15).

### FIELD OF THE INVENTION

The present invention relates generally to a blood pumping system and procedure for supplementing blood circulation, in particular, to a system which supplements blood perfusion in distal regions of the patient's circulatory system at normal and/or elevated pressures.

### BACKGROUND OF THE INVENTION

There has been a pressing need for a device that is capable of supplementing blood perfusion in distal parts of a patient's circulatory system. It is common in patients suffering from Congestive Heart Failure (“CHF”), diabetes, gangrene, erectile dysfunction, atheroma or obstructive vessel disease to also suffer from poor circulation. Poor circulation often leads to a patient suffering from ischaemia or chronic oxygen debt in distal regions of their circulatory system. It is this ischaemia or oxygen debt that can lead to exercise related claudication or rest pain. These diseases may also lead to various other complications that may require radical surgery to correct, such as amputation of limbs. The present invention seeks to address these problems.

Traditionally, poor circulation in limbs has been treated by a wide range of devices and procedures, which were aimed at promoting blood flow through a limb and by doing so, induce revascularisation of the limb.

In the past, hyperbaric chambers have been used to increase oxygen tension within the limbs. This increases oxygen tension delivered to distal regions of the patient's circulatory system and leads to a reduction of oxygen debt in these regions. Hyperbaric chambers are often expensive, inefficient, reduce the patient's quality of life and are suitable only for short term use.

It is an object of the present invention to address or ameliorate one or more of the abovementioned disadvantages and to address the problem of supplementing blood perfusion in at least a distal region of the patient's circulatory system.

### BRIEF DESCRIPTION OF INVENTION

Accordingly to a further broad form of the present invention, there is provided a blood pumping system, for supplementing distal blood perfusion, comprising: a blood pressure altering device including an upstream end and a downstream end; wherein said blood pressure altering device cooperates with a circulatory system to promote blood flow throughout at least one or more distal regions including limbs, a brain region or a pelvic region of a patient by altering vascular blood pressure; whereby said blood pressure altering device, when in use, is positioned in series with the normal blood flow of the circulatory system of a patient.

Preferably, the blood pressure altering device, when in use, increases blood pressure in a localised region and the blood pressure altering device may also be a blood pump.

Preferably, the system provides a means of vascular regeneration, when in use.

Preferably, the blood pressure altering device may also be a blood pump. The blood pump may comprise an inlet and an outlet for connection to the circulatory system of a patient. The inlet may also be connected to an arterial system of the patient or the inlet may be connected to a venous system of the patient. The outlet may be connected to an arterial system of the patient. The inlet or outlet may also include cannula extensions to allow variable positioning of the blood pump. The preferred blood pump is also implantable within the body of a patient. Said system may also include an implantable power source and implantable controller both to cooperate with said blood pump.

Preferably, the blood pump has a relatively flat H-Q curve characteristic and is capable of delivering a relatively constant mean blood pressure and said blood pressure may be relatively accurately determined by pump speed without the need for an implanted sensor.

The system may include a flow-back shunt to allow blood to flow from an outlet of the blood pump back to an inlet of the blood pump. Said flow-back shunt may include a flow resistor that is capable of restricting blood flow through flow-back shunt. Said flow resistor may be regulated externally relative to the patient. Said system may also include at least one fistula, when in use, connected between the outlet and a desired site in the venous system to allow blood flow communication between said outlet and said site in the venous system. Said fistula may include a variable regulator for controlling blood rate within said fistula.

Said system may supplement distal blood perfusion on either a short term basis or a long term basis.

Said blood pump may also be positioned external to the body of the patient.

It is preferred that said system may also be supplemented by a regime of pharmaceuticals given to the patient that promote revascularisation of distal regions of blood circulation. Said system may be supplemented by a regime of pharmaceuticals given to the patient that promote vascular dilatation of distal regions and neovascularisation of patient's circulatory system.

Preferably, the circulatory system may be reinforced with stenting.

The preferred system may include at least one sensor to measure effectiveness of supplementing distal blood perfusion. The blood pressure altering device may include at least one additional outlet for connection to a haemodialysis system.

Accordingly to a further broad form of the present invention, there is provided a blood pumping system, for perfusing a distal region of a patient's circulatory system, comprising: a blood pressure altering device, wherein said blood pressure altering device is in fluid communication with said circulatory system, and wherein said blood pressure altering device pumps blood so as to create a localised hypertensive region in said distal region. Preferably, said distal region includes a portion of the arterial blood supply of the circulatory system.

Preferably, said blood pressure altering device is located in a position remote from the heart of the patient.

It is preferable for said system supply a continuous supra-systolic pressure in both systole and diastole and that said blood pressure altering device may be positioned in series with the normal blood flow of a circulatory system.

Preferably, said localised hypertensive region may be created downstream from the blood pressure altering device. Additionally, the blood pressure altering device may be a pump. Said pump may be implantable within the body of a patient and have a relatively flat flow pressure curve characteristic.

Preferably, said system provides a means of vascular regeneration and may also include a flow resistor.

### BRIEF DESCRIPTION OF THE DRAWINGS

Embodiments of the invention will now be described with reference to the drawings in which:

FIG. 1 is a cross-sectional side view of a patient implanted with a first preferred embodiment;

FIG. 2 is a cross-sectional side view of a patient implanted with a second preferred embodiment;

FIG. 3 is a cross-sectional side view of a patient implanted with a third preferred view; and

FIG. 4 is a cross-sectional view of a patient implanted with a fourth preferred embodiment.

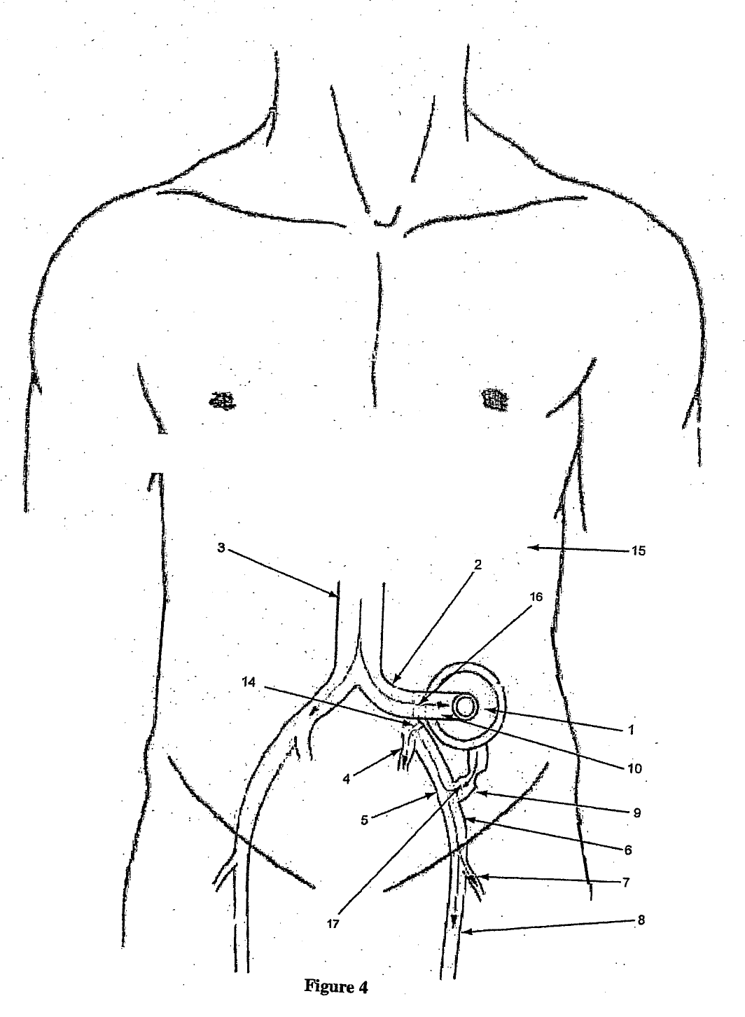

### DETAILED DESCRIPTION OF PREFERRED EMBODIMENTS

The aim of the embodiments of the present invention is to increase the input blood pressure to a limb of a mammal, usually a human patient. By locally increasing the blood pressure, a greater pressure gradient is generated in a distal region of the patient's circulatory system (in this embodiment the distal region is a limb of the patient 15) and thereby greater blood flow is encouraged within the limb. Increased local blood pressure also increases the partial pressures of the dissolved oxygen leading to better tissue nutrition. The oxygen debt and toxic metabolites that have accumulated are therefore decreased.

This increased perfusion is intended to promote healing, encourage development of co-lateral vessels and may reduce other associated symptoms, such as ulceration and pain. The aim is to create a localised area of hypertension at the upper part of the limb arterial system to reverse ischemic changes.

In the lower limbs of a human patient there are three distal compartments. These are the anterior tibial, posterior tibial and peroneal compartments. In peripheral vascular disease, there are different numbers and sizes of collaterals. Embodiments of the present invention achieve this by locally increasing the blood pressure and thereby increasing blood flow throughout the largest cross compartment collaterisation. This effect also may increase with increased perfusion pressure generated by the embodiment.

Preferably, an optimal collateral flow generally occurs at steady input pressures. The preferred embodiments of the present invention may increase in all three parameters; ie: systolic, mean and diastolic pressures are sought to be achieved.

An increase in the mean blood pressure within a limb or distal region of the circulatory system may improve neovascularisation. Preferred embodiments of the present invention may create localised areas of hypertension and thereby vascular neogenesis is stimulated, resulting in an improved blood flow circulation throughout the limb.

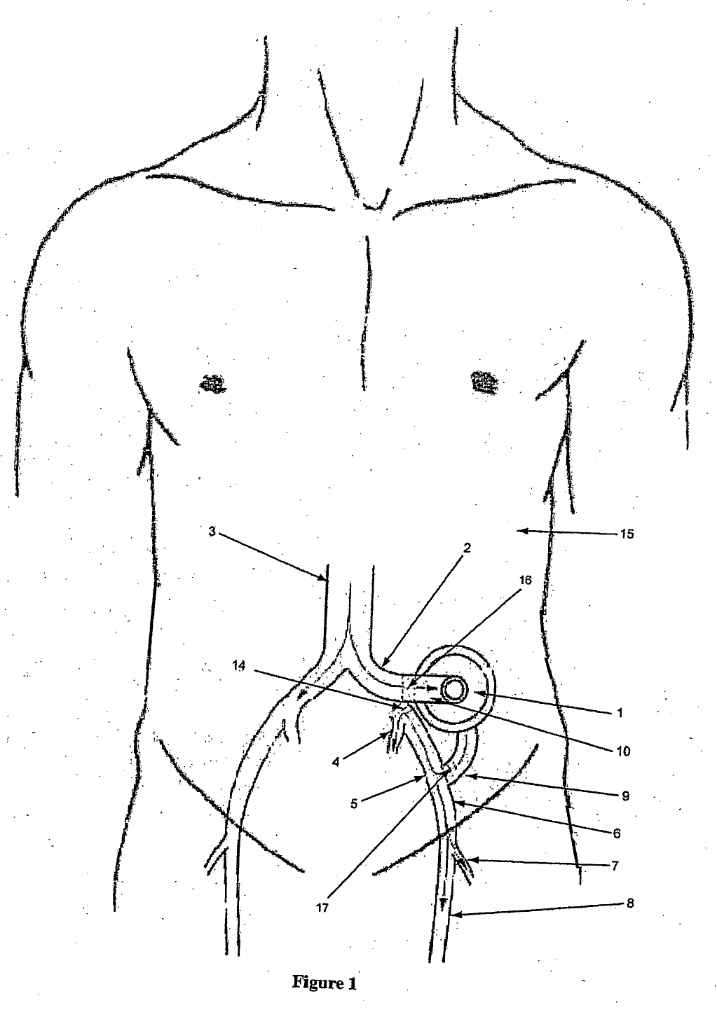

The first preferred embodiment of the invention applied to a human patient 15 is shown in FIG. 1. In this embodiment, a blood pressure altering device is implanted in series to the normal blood pressure path throughout the circulatory system of a patient 15. In particular, this embodiment shows the blood pressure altering device located in series with the arterial blood path to increase blood pressure and flow in the left leg of the patient 15. The blood pressure altering device, in this configuration, operates and cooperates in series with the existing blood path of the patient to pressurise the blood in the localised area. This may create a region of localised hypertension directly adjacent to the downstream area of the blood pressure altering device.

In this preferred embodiment of the present invention, the blood pressure altering device may be a blood pump 1. Specifically, the blood pump 1 may be a centrifugal rotary blood pump, which is implantable within the body of a patient 15. The preferred blood pump may also have a relatively flat H-Q curve to allow the pressure output to be accurately determined from only the power inputted into said pump 1.

In FIG. 1, blood pump 1 has an inlet 10 at the upstream portion of the blood pump and outlet 9 positioned at the downstream portion of the pump. Typically, blood pump 1 is constructed so as to allow blood flow through the device. The pumping action of the blood pump 1 produces a localised region of hypertension within the circulatory system of the patient directly adjacent to downstream region of blood pump 1. This localised hypertension is sufficient to increase blood flow in body regions adjacent to the blood pump 1 and in distal regions of the limb. Please note that “distal” in relation to this specification, means situated away from the patient's heart.

Also, in this preferred embodiment of the present invention, the common iliac artery 2, which is downstream of the abdominal aorta 3, of patient 15 is severed at position 14. The inlet 10 of said pump 1 is then connected to the upstream portion of the common iliac artery 2 by connection means 16. This connection means 16 is preferably accomplished by either use of sutures, expanding stents, bio-glue and or adhesive.

Additionally, the downstream portion of the blood pump 1 has an outlet 9. In this preferred embodiment, this outlet 9 is connected to an external iliac artery 5 by connection means 17. This connection means 17 is preferably accomplished by the use of sutures, expanding stents, bio-glue and/or adhesive.

The severance of artery 2 at position 14 results in two ends of the artery being formed. In this preferred embodiment, as earlier stated, the upstream end is connected to pump 1. The downstream end of the artery, adjacent to the internal iliac artery 4, is not connected and left as a free end. This free end should be sealed to prevent blood leakage into internal body cavities of the patient.

In this preferred embodiment of the present invention the artery delivers blood to blood pump 1 and the blood pump pressurises the blood into artery 5, which in turn may increase the blood pressure and blood flow in the profunda femoris artery 7, common femoris artery, superficial femoral artery 8, internal iliac artery 4 and the external iliac artery 5. In doing so, this preferred embodiment induces a localised hypertensive state in at least a portion of the desired limb. In FIG. 1, the desired limb in which the hypertensive state is induced is the patient's left leg. In other embodiments of the present invention, the pump 1 may be connected in such a manner so as to induce a localised hypertensive state in other limbs or other distal regions of the body including pelvis regions, and/or brain.

Additionally, it is envisaged that inlet 10 and outlet 9 may include an expandable stent which may allow the pump to be positioned at a different site in the body rather than proximal to artery 2 and 5. Also, it is envisaged that connections sites between the inlet 10 and the artery 2 and outlet 9 with artery 5 can be reinforced by stenting the artery to prevent collapse or rupture. This is particularly relevant as the increased blood flow and pressure in the localised area may cause either one of these situations to occur.

Please note that in the embodiment shown in FIG. 1, the pump 1 is located remote from the patient's heart. In the context of this specification, remote from the patient's heart essentially means that the pump 1 is not implanted in the chest cavity of the patient to be treated.

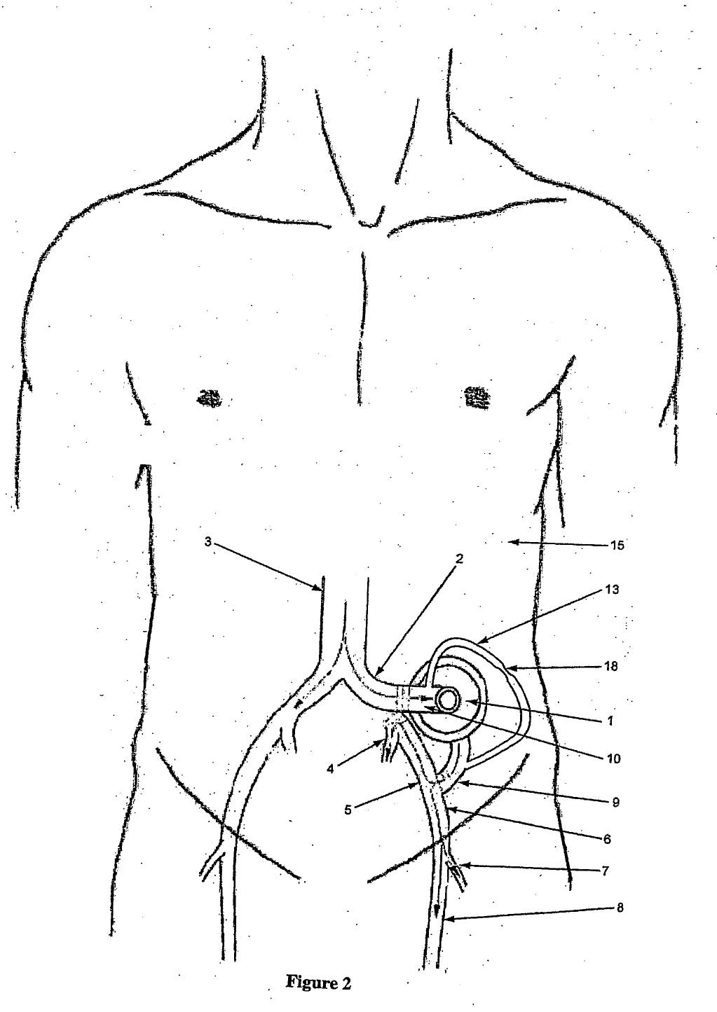

In FIG. 2, an alternative preferred embodiment of the present invention is shown wherein a flow back shunt 13 is inserted into the patient's body 15 and connected to the inlet 10 and outlet 9 to allow the circular blood flow communication around the blood pump 1. The effect of this flow back shunt 13 is to prevent excessive or undesired blood pressure from entering the patient's circulatory system. The flow back shunt 13 splits the blood flow in the outlet 9 and redistributes the excessive flow back to inlet 10.

Preferably, said flow back shunt 13 includes a flow back resistor 18 that is capable of restricting the blood flow through said flow back shunt 13. This resistor 18 preferably may be regulated by an external means.

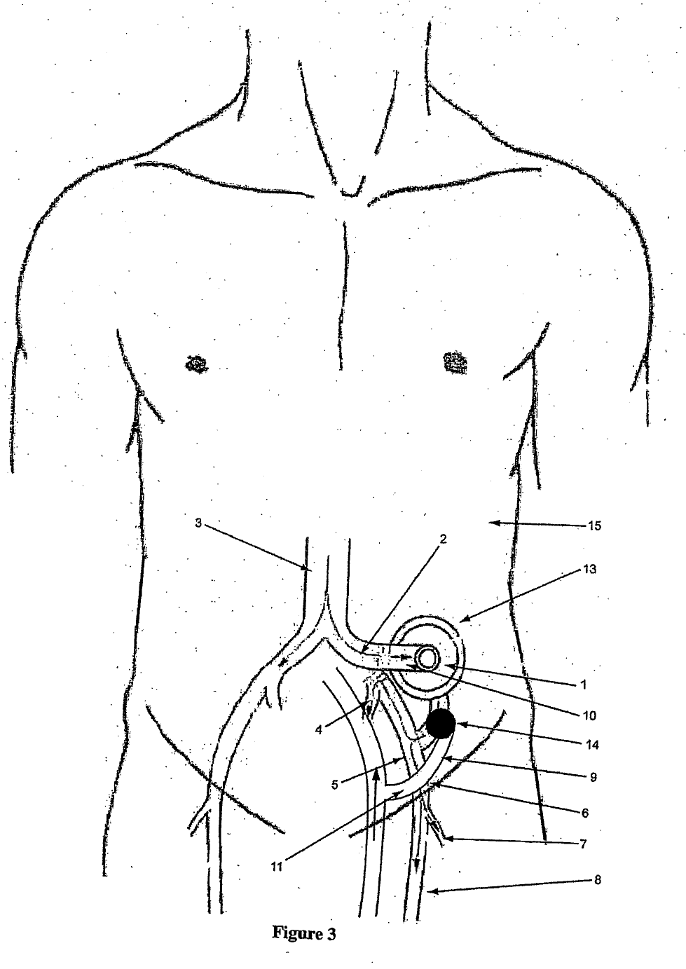

FIG. 3 shows an application of a third preferred embodiment of a present invention. In this preferred embodiment a fistula 11 is added to the system. This fistula 11 is connected between the venous system and the outlet 9. The fistula 11 allows blood communication from the outlet of the pump directly to the vein assistance and thereby allowing the blood to bypass the limb in the event of excessive pressure being built up within the downstream arteries of the leg.

Preferably, the blood flow through fistula 11 may be controlled by a variable regulator 14. Thereby the variable regulator 14 may control the reduction of undesirably high blood pressures.

Embodiments of the present invention may also include a blood pump that is attached to a venous system of the patient. The result would be that the change in blood pressure caused by the blood pump connected to circulatory system may pull blood through the limb as opposed to the aforementioned preferred embodiments that push the blood through the circulation system within the limbs by increasing blood pressure.

Also, multiple blood pumps may be implanted within a patient's circulatory system to allow multiple regions of localised hypertension within the patient.

Additionally, it may be beneficial to include within any of the preferred embodiments at least one sensor to detect and record blood pressure, blood flow or status information relating to the pump 1. Embodiments of the present invention may also include: a blood pump with multiple outlet which may be connected to arteries supplying respective multiple limbs.

Embodiments of the present invention may also be supplemented by the use of an appropriate regime of pharmaceuticals that promote vascular dilation of the distal regions of the circulatory system.

It is important to note that the present invention may function in a similar way if the pumping system was arranged to supplement blood flow in other distal regions of the circulatory system such as the arms, the legs, the pelvic region and/or the brain region.

Another preferred embodiment of the present invention includes a blood hyper-perfusion pumping system for supplementing flow, wherein said system creates a lower relative mean blood pressure at a cardiac side of a pumping device and a higher relative mean blood pressure at a distal ischaemic side. This system preferably is connected to the patient's circulatory system on the arterial side of said system.

Various modifications and alterations are possible within the spirit of the foregoing specification and drawings without departing from the scope of this invention.

1. A blood pumping system, for supplementing distal blood perfusion, comprising: a blood pressure altering device including an upstream end and a downstream end; wherein said blood pressure altering device cooperates with a circulatory system to promote blood flow throughout at least one or more distal regions including limbs, a brain region or a pelvic region of a patient by altering vascular blood pressure; whereby said blood pressure altering device, when in use, is positioned in series with the normal blood flow of the circulatory system of a patient.

2. The system as in claim 1 wherein the blood pressure altering device, when in use, increases blood pressure in a localised region.

3. The system as in claim 2 wherein the blood pressure altering device is a blood pump.

4. The system as in claim 2 wherein the system provides a means of vascular regeneration, when in use.

5. The system of claim 3 wherein the blood pump comprises an inlet and an outlet for connection to the circulatory system of a patient.

6. The system of claim 5 wherein the inlet is connected to an arterial system of the patient.

7. The system of claim 5 wherein the inlet is connected to a venous system of the patient.

8. The system of claim 7 wherein the outlet is connected to an arterial system of the patient.

9. The system of claim 5 wherein the inlet or outlet includes cannulae extensions to allow variable positioning of the blood pump.

10. The system as in claim 5 wherein the blood pump has a relatively flat H-Q curve characteristic.

11. The system as in claim 5 wherein delivered blood pressure is relatively constant and is relatively accurately determined by pump speed without the need for an implanted sensor.

12. The system as in claim 5 wherein said system includes a flow-back shunt to allow blood to flow from an outlet of the blood pump back to an inlet of the blood pump.

13. The system as in claim 12 wherein said flow-back shunt includes a flow resistor that is capable of restricting blood flow through flow-back shunt.

14. The system as in claim 13 wherein said flow resistor is regulated externally.

15. The system as in claim 5 wherein said system includes at least one fistula, when in use, connected between the outlet and a desired site in the venous system to allow blood flow communication between said outlet and said site in the venous system.

16. The system as in claim 15 wherein said fistula includes a variable regulator for controlling blood rate within said fistula.

17. The system as in claim 1 wherein said system supplements distal blood perfusion on a short term basis.

18. The system as in claim 1 wherein said system supplements distal blood perfusion on a long term basis.

19. The system as in claim 17 wherein said blood pressure altering device is a blood pump and is implantable.

20. The system as in claim 19 wherein said system includes an implantable power source and implantable controller both to cooperate with said blood pump.

21. The system as in claims 17 wherein said blood pressure altering device is a blood pump that is external to the body of the patient.

22. The system as in claim 17 wherein said system is supplemented by a regime of pharmaceuticals given to the patient that promote revascularisation of distal regions of blood circulation.

23. The system as in claim 17 wherein said system is supplemented by a regime of pharmaceuticals given to the patient that promote vascular dilatation of distal regions and neovascularisation of patient's circulatory system.

24. The system as in claim 17 wherein the circulatory system is reinforced with stenting.

25. The system as in claim 17 wherein said system includes at least one sensor to measure effectiveness of supplementing distal blood perfusion.

26. The system as in claim 17 wherein said blood pressure altering device includes at least one additional outlet for connection to a haemodialysis system.

27. (canceled)

28. A blood pumping system, for perfusing a distal region of a patient's circulatory system, comprising: a blood pressure altering device, wherein said blood pressure altering device is in fluid communication with said circulatory system, and wherein said blood pressure altering device pumps blood so as to create a localised hypertensive region in said distal region.

29. The system as claimed in claim 28, wherein said blood pressure altering device is located in a position remote from the heart of the patient.

30. The system as claimed in claim 28, wherein said distal region includes a portion of the arterial blood supply of the circulatory system.

31. The system as claimed in claim 28, wherein said system supplies a continuous supra-systolic pressure in both systole and diastole.

32. The blood pumping system as claimed in claim 28, wherein said blood pressure altering device is positioned in series with the normal blood flow of a circulatory system.

33. The blood pumping system as claimed in claim 28, wherein said localised hypertensive region is created downstream from the blood pressure altering device.

34. The blood pumping system as claimed in claim 28, wherein the blood pressure altering device is a pump.

35. The blood pumping system as claimed in claim 28, wherein said system provides a means of vascular regeneration.

36. The blood pumping system as claimed in claim 28, wherein said blood pumping system includes a flow resistor.

37. The blood pumping system as claimed in claim 28, wherein said blood pressure altering device is implantable within the body of a patient.

38. The blood pumping system as claimed in claim 28, wherein said blood pressure altering device has a relatively flat flow pressure curve characteristic.
